(19) United States
(12) Patent Application Publication
(10) Pub. No.: US 2007/0282184 A1
(43) Pub. Date: Dec. 6, 2007

(54) Bladder Mapping

(76) Inventor: Paul R. Roberts, Hampshire (GB)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/596,934

(22) PCT Filed: Jan. 11, 2005
(86) PCT No.: PCT/GB05/00056

§ 371 Date: May 31, 2007

(30) Foreign Application Priority Data
Jan. 13, 2004 (GB) 0400687.0

(51) Int. Cl. A61B 5/05

(52) U.S. Cl. 600/345

(57) ABSTRACT

A device is provided for detecting the location of electrical activity in the wall of the human bladder. In a preferred embodiment the device is insertable through the urethra, and is expandable. The device may further include an ablation tool for ablating the interior wall of the bladder at a site of electrical activity.

This invention relates to human bladder mapping, and to apparatus for treatment of the human bladder.

As is well-known, the human bladder accumulates urine for periodic release at a convenient place and time. Instability of the bladder may result in uncontrolled leakage, so called stress incontinence, which can be both unpleasant and embarrassing. Instability may be caused for example by infection, a poorly functioning sphincter, or by spasm of the bladder.

In the latter case it is known that the bladder wall is a focal area of electrical activity within the human body, and that abnormal electrical bursts may occur. This electrical activity, which cannot be felt, is considered to be one cause of bladder instability.

According to a first aspect of the invention, there is provided apparatus for detecting the location of electrical activity in the wall of a human bladder.

Upon detection of electrical activity, a treatment of the location may prevent subsequent activity, or may mitigate the effect thereof.

Detection apparatus may be external or internal to the human body, and comprise for example a spaced array of devices adapted to detect electrical activity, and to permit the location of said activity to be determined, for example by mathematical techniques such as triangulation.

In the case of external apparatus, the devices may be arranged for example on or in a flat support for the body, and in a triangular formation; such a flat support is typically a bed on which a patient may lie so that the bladder is at a determinable distance from the detection device.

The apparatus may however comprise a detector adapted to be introduced into the bladder via the urethra, and having a connector to the exterior whereby the location of electrical activity in the wall of the bladder can be determined. Insertion through the urethra does not require any surgical intervention, and is thus a convenient and safe method of obtaining direct detection of electrical activity.

The detector may for example be a contact device adapted to touch the interior wall of the bladder and thereby detect electrical activity. In one embodiment the device comprises a cage having a plurality of detection sites thereon, the cage being insertable through the urethra in a collapsed form and being expandable within the bladder into contact with the wall thereof. A suitable reversible expanding mechanism may rely upon relative telescopic movement of inner and outer members of connector passing to the exterior. In a preferred embodiment the cage is open so that expansion and contraction is unimpeded by fluid within the bladder.

In use such a device contacts the wall of the bladder at a plurality of places. Electrical activity within the bladder is detected at one or more detection sites, and information concerning the location of this activity passes to the exterior via the connector. Each detection site may for example be adapted to sense a transient voltage, and thus to indicate a location or path of electrical activity by the size of the voltage detected at one or more sites. Where several sites detect substantially simultaneous electrical activity, triangulation techniques may be used to determine the location of the source.

An expandable detector is typically a regular three-dimensional body in the expanded state, and has uniformly distributed detection sites thereon. In one embodiment the detector resembles a sphere in the expanded state, and has a plurality of arcuate arms extending between opposite poles, one of which poles is coupled to the exterior connector. In the collapsed state the arms lie in close proximity to each other so as to have a small overall cross-section and thus be adapted for insertion through the urethra.

The expanded envelope of such a device is preferably selected to suit the approximate size of the bladder into which it is to be inserted. However it is also envisaged that the bladder may itself be stretched by the device so as to ensure more uniform contact by the detection sites thereof.

The number of detection sites are selected to allow the location of electrical activity to be determined with reasonable certainty. In the case of the spherical device noted above, eight equispaced arms may be provided, each with eight equispaced detection sites thereon. The greater the number of detection sites, the more precise can be location of the source.

The apparatus may include further means to permit the orientation thereof to be determined. Such means are not essential, as will become apparent, but may be useful in determining the actual location of electrical activity in relation to other body organs. Approximate orientation may be determinable from the connector, and for example a longitudinally extending mark thereon. Orientation means may also comprise for example one or more identifiable detection sites adapted to be stimulated from the exterior, or an asymmetric array of detection sites.

In a preferred embodiment said connector may include one or more lumens to permit access from the exterior. A lumen may for example permit the bladder to be filled with liquid so as to become distended. A distended bladder may be more suitable for application of the detector, and is also more likely to stimulate bursts of electrical activity.

The same or another lumen may be used for the introduction of medicaments.

The connector is preferably relatively stiff so as to permit easy insertion thereof through the urethra and to permit some steering of the detector. However a lumen of the connection may be adapted to take a relatively stiff guide member so as to ease passage thereof. The guide member may be curved so as to place the detector in a desired position within the bladder.

Where lumens are provided for fluid, suitable valve means are typically required so as to prevent voiding of the bladder. Such valve means may be at the exterior in the form of e.g. a tap or clamp, or may be at the interior in the form of e.g. a self closing slit diaphragm.

The apparatus may further include a tubular introducer via which the device may be inserted into the bladder. Such an introducer may itself be inserted after enlargement of the urethra by a dilator, and typically has an exterior valve to prevent voiding of the bladder when in place. The introducer may have one or more side ports adapted to permit insertion of the apparatus therethrough, and other tubes or guides, as may be required.

In an alternative embodiment the detector may be of the non-contact kind adapted to detect electrical activity in the wall of the bladder whilst being located within the bladder at a distance from the wall thereof. As will be appreciated such a non-contact device may be somewhat smaller than a contact type and thus less difficult to pass through the urethra. A suitable non-contact type may be spherical. Like the contact type described above eight equispaced arms may connect the poles, each arm having eight detection sites thereon. Being smaller, this non-contact detector may comprise a balloon insertable in the deflated state, and inflatable by injection of fluid through a connecting lumen. Typical inflation means comprise a syringe for injecting a precise volume of physiologically acceptable solution into the balloon.

In order to maintain such a balloon or other non-contact detector at the approximate centre of the bladder, a stiff or stiffened connector is preferably used. Alternatively the non-contact detector may be provided with one or more spacing members on the surface thereof and movable from a stowed to a deployed condition. In the case of an inflatable detector, the spacing members may deploy automatically as inflation occurs, and be adapted to restow as the detector is deflated, or as it is withdrawn from the bladder.

The spacing members may for example comprise arms or loops on the surface of an inflatable detector and adapted to stand up on inflation, or may themselves each comprise an inflatable soft tube.

The spacing members need not all contact the wall of the bladder provided that the detector is stabilised.

The detection apparatus may be passive in the sense that it acts as an antenna capable of detecting electrical activity at a distance. Alternatively the detection apparatus may include transmission apparatus adapted to generate a field in the vicinity of the bladder, and detectors adapted to detect distortions of the field which are a consequence of electrical activity in the wall of the bladder.

Apparatus according to the invention may further include a device for imaging the interior wall of the bladder, such as by ultrasound. In this way the user may be able to correlate a physical feature of the bladder wall with a site of electrical activity.

According to a second aspect of the invention there is provided a method of introducing the detector into a human bladder comprising the steps of contracting the detector into a form having a substantially cylindrical envelope, introducing the detector through the urethra by means of a connector attached to the detector, and expanding the detector in the bladder by means of said connector. Means for expanding the detector may be mechanical, such as relatively telescopic connector components or fluid actuated, for example by introduction of liquid into an inflatable member.

According to a third aspect, the invention provides apparatus for ablation of the bladder from the interior thereof, and comprising an ablation tool adapted to be introduced into the bladder via the urethra, and operable on command to ablate the wall of the bladder. The ablation tool may for example comprise an energy source relying on heat (radio frequency or laser) or cold (cryogenic).

Such ablation apparatus acts by disrupting the surface of the muscle so as to change the response thereof to bursts of electrical activity. The ablation may for example scar the surface of the bladder wall so as to prevent electrical impulses following an undesirable path.

Suitable ablation apparatus may comprise a relatively stiff wire having a bend at the distal end, and an ablation tool at the tip. After insertion into the bladder, the tip is adapted to be steered internally to any desired region by the use of external control apparatus of a conventional kind. Such apparatus may rotate the wire, and change the insertion depth.

The tip of the ablation apparatus is preferably adapted to be detectable, for example by being electrically active, whereby its location can be detected by a suitable detector in the vicinity of the bladder. Accordingly the ablation tool can be maneuvered to the site of electrical activity within the bladder, and operated to ablate the site. The tip of the ablation apparatus may be detectable externally, for example by CT imaging.

In a preferred embodiment however, an ablation tool is used in conjunction with apparatus for detecting electrical activity in the bladder so as to treat bladder instability. The ablation tool is preferably introduced along the connector of an internal apparatus, and preferably within a lumen thereof, so that the detection apparatus both detects electrical activity of the bladder, and of the ablation tool.

Other features of the invention will be apparent from the following description of a preferred embodiment shown by way of example only in the accompanying drawings, in which:

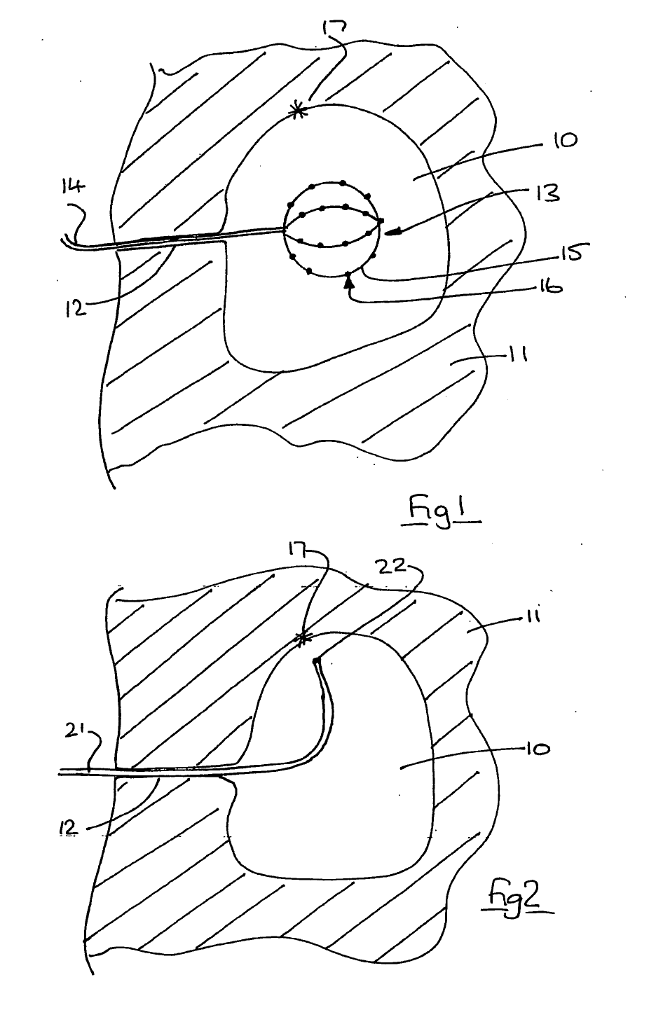

FIG. 1 is a schematic illustration of a detector of the invention;

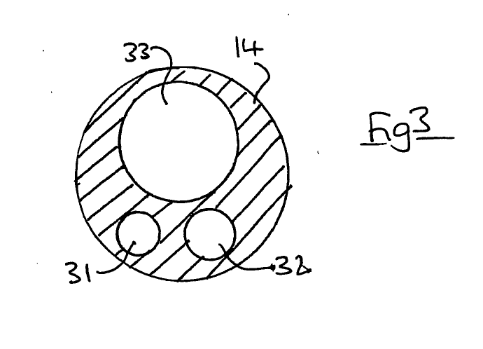

FIG. 2 is a schematic illustration of an ablation tool; and

FIG. 3 is a cross-section through a connector for the detector of the invention.

FIG. 1 shows schematically a somewhat distended bladder 10 connected to the exterior of the human body 11 by a urethra 12. An inflatable detector 13 is passed through the urethra in a collapsed state, and inflated (as illustrated) when in the bladder by injection of a precise volume of saline solution.

The detector 13 is coupled to a relatively stiff connector 14 which incorporates an inflation lumen and permits the position of the detector to be adjusted. In use the detector is positioned substantially at the 3-dimensional centre of the bladder as illustrated, at a spacing of 4-5 cm from the bladder wall.

The surface of the detector 13 comprises a series of equispaced wires 15 coupled at the poles. Each wire has four detection sites 16 thereon, which may be formed by laser removal of insulation. The sensitivity and range of the detector may of course require adjustment using conventional techniques, and is according to the nature and size of the bladder.

Thus a burst of electrical activity 17 is in use detected by several sites 16, and computer techniques are used to identify the location of the burst, by reference to, for example, the relative strength of the signal detected at each of several sites. Suitable electrical connections to the exterior are provided via the connector 14. The sites 16 may also be adapted to detect the location of electrical tracking along and through the bladder wall, by for example continual real time sampling of the sites 16.

Mapping of electrical activity is a useful precursor to diagnosis and treatment of bladder instability.

FIG. 2 illustrates a bladder 10 into which an ablation device 21 is introduced via the urethra 12. The device typically has a small r.f. heat source 22 at the tip thereof, and can be actuated to scar the site of a burst of electrical activity 17.

In order to position the heat source 22, the device 21 can be moved back and forth in the urethra, and rotated. It may also be possible to bend and unbend the distal portion, using known techniques, so as to ensure that the entire surface of the bladder wall can be reached. In practice the tip 22 may be adapted to emanate an electrical signal, and the device 21 may be used in conjunction with the detector 13 so as to locate the tip accurately on the bladder wall.

Bursts or single spikes of electrical activity can occur spontaneously, and as the bladder is stretched. Accordingly in a preferred embodiment of the invention a lumen is provided whereby the bladder can be filled via the invention in order to stimulate electrical activity for diagnosis and treatment.

Typically a volume of physiologically acceptable liquid, such as saline solution, is passed through the urethra in a controlled manner for example from an elevated reservoir, and via a one-way valve at the exterior side. The lumen may be incorporated in the connector 14, or may lie at the side thereof. It will be understood that the detector is activated during filling of the bladder so as to detect electrical activity as the bladder wall is stretched.

FIG. 3 illustrates how the cross section of a connector 14 may appear in the preferred embodiment, and having an electrical connection 31 for transmitting mapping information, an inflation lumen 32 for the detector, and a filling lumen 33 for saline solution. The inflation lumen is necessarily closed at the distal end by the balloon, but the filling lumen 33 is open. The filling lumen may pass through the balloon to the distal side, and thus provide for passage of a removable guide wire to stiffen the connector/balloon during insertion thereof. Such a guide wire preferably has a conventional pigtail end to prevent accidental damage to the bladder wall as the device is inserted through the urethra.

At the exterior side such a combination lumen includes suitable closures, such as valves, to maintain the balloon in an inflated state, and to maintain the bladder in an expanded state. Release of these valves respectively allows the balloon to deflate for withdrawal, and for the bladder to drain.

The filling lumen 32 may also be used to transfer a medicament into the bladder, for example through injection via an exterior side port.

1-10. (canceled)

11. Apparatus for detecting the location of electrical activity in the wall of a human bladder.

12. Apparatus according to claim 11 and comprising an expandable device adapted for passage through the urethra in a collapsed condition and reversibly expandable when in the bladder.

13. Apparatus according to claim 12 wherein said expandable device has a plurality of detection sites thereon.

14. Apparatus according to claim 13 wherein said detection sites are uniformly distributed on the surface thereof.

15. Apparatus according to claim 14 wherein said expandable device resembles a sphere in the expanded state,

16. Apparatus according to claim 14 wherein said expandable device comprises a cage having a plurality of arcuate arms extending between opposite poles.

17. Apparatus according to claim 12 and including an external telescopic connector whereby relative telescoping movement causes the device to expand and contract on demand.

18. Apparatus according to claim 12 and comprising an inflatable device.

19. Apparatus according to claim 18 wherein said device includes an inflation lumen having an external closure.

20. Apparatus according to claim 12 and further including a fluid lumen adapted to permit filling of the bladder from the exterior.

21. Apparatus according to claim 20 and comprising multiple lumens.

22. Apparatus according to claim 11 and comprising an array of detection sites adapted to detect electrical activity in the wall of the bladder whereby the location of said electrical activity can be determined.

23. Apparatus according to claim 22 wherein said detection sites are uniformly distributed.

24. Apparatus according to claim 12 and further comprising orientation means whereby the orientation of an expandable device in the bladder may be determined from outside the bladder.

25. Apparatus according to claim 12 and further including a lumen adapted to receive a stiff curved guide member for steering of the expandable device.

26. Apparatus according to claim 11 and further including an ablation tool adapted for insertion through the urethra and operable to ablate the internal surface of the bladder wall.

27. Apparatus according to claim 26 wherein the tip of said tool is detectable by position sensing apparatus.

28. Apparatus according to claim 27 wherein the tip of said tool is adapted to be electrically active and wherein said apparatus is adapted to detect said activity.
